(19) United States
(12) Patent Application Publication
(10) Pub. No.: US 2023/0172487 A1
(43) Pub. Date: Jun. 8, 2023

(54) DROWSINESS WARNING SYSTEM AND METHOD

(71) Applicant: SAMSUNG LIFE PUBLIC WELFARE FOUNDATION, Seoul (KR)

(72) Inventors: Moon Ki CHAE, Seoul (KR); Dong Yeol KWON, Gyeonggi-do (KR)

(73) Assignee: SAMSUNG LIFE PUBLIC WELFARE FOUNDATION, Seoul (KR)

(21) Appl. No.: 17/998,727

(22) PCT Filed: May 14, 2021
(86) PCT No.: PCT/KR2021/006052

§ 371 Date: Nov. 14, 2022

(30) Foreign Application Priority Data
May 15, 2020 (KR) 10-2020-0058365

(51) Int. Cl. A61B 5/11; A61B 5/00

(52) U.S. Cl. CPC A61B 5/11; A61B 5/6821; A61B 5/4812; A61B 5/746; A61B 5/486

(57) ABSTRACT

A drowsiness warning system is disclosed. Specifically, the drowsiness warning system includes a first sensor unit configured to be mountable on any one of upper eyelids or lower eyelids of a subject being examined under treatment by using treatment equipment, a second sensor unit configured to be mountable on the other one of the upper eyelids or the lower eyelids of the subject being examined, an output unit configured to generate a drowsiness preventing signal for preventing drowsiness of the subject being examined, and

a control unit configured to determine a drowsy state of the subject being examined, based on a distance between the first sensor unit and the second sensor unit, wherein, when the distance is maintained for a predetermined time or more in a state of being shorter than a predetermined distance, the control unit is further configured to determine that the subject being examined is in the drowsy state, and control the output unit to transmit a drowsiness preventing signal to the subject being examined.

### TECHNICAL FIELD

The present disclosure relates to a system and method for preventing and warning drowsiness of a subject being examined.

### BACKGROUND ART

Recently, due to the development of treatment technologies, gating treatment of SBRT (Stereotatic Body Radiation Therapy) or IMPT (Intensity Modulated Proton Therapy) has been performed. Long treatment times (up to 1 to 2 hours) often cause drowsiness in patients, which may further delay the treatment time or lead to inaccurate treatment. Accordingly, there is a need for a method for enabling a smooth treatment by preventing drowsiness and a decrease in concentration of a patient.

Currently, there is no way to check the drowsiness of the patient. And thus, a monitoring device such as an infrared camera is used to observe the breathing pattern of the patient's respiratory cycle. And based on the subjective judgment of monitoring staff, if an abnormality is found in the breathing pattern of the patient, an announcement is broadcast through a microphone connected to the inner area of the treatment room to wake the patient. However, in reality, there is no objective system or method for judging the drowsiness state of a patient.

### DESCRIPTION OF EMBODIMENTS

### Technical Problem

The present disclosure provides a drowsiness warning system and method capable of preventing drowsiness of a subject being examined under treatment for a long time.

### Solution to Problem

According to an aspect of the present disclosure, a drowsiness warning system includes a first sensor unit configured to be mountable on any one of upper eyelids or lower eyelids of a subject being examined under treatment by using treatment equipment, a second sensor unit configured to be mountable on the other one of the upper eyelids or the lower eyelids of the subject being examined, an output unit configured to generate a drowsiness preventing signal for preventing drowsiness of the subject being examined, and

a control unit configured to determine a drowsy state of the subject being examined, based on a distance between the first sensor unit and the second sensor unit, wherein, when the distance is maintained for a predetermined time or more in a state of being shorter than a predetermined distance, the control unit is further configured to determine that the subject being examined is in the drowsy state, and control the output unit to transmit a drowsiness preventing signal to the subject being examined.

The control unit may be connected to the treatment equipment to stop an operation of the treatment equipment when it is determined that the subject being examined is in the drowsy state.

The drowsiness warning system may further include a notification unit configured to notify an examiner of the drowsy state of the subject being examined, wherein when it is determined that the subject being examined is in the drowsy state, the control unit is further configured to control the notification unit to generate a drowsiness warning signal for notifying the examiner.

The drowsiness warning signal may include at least one of a first signal including a sound signal, a second signal including an optical signal, or a third signal including a vibration signal.

The output unit may further configured to transmit the first to third signals to the subject being examined in a stepwise manner.

The output unit may further configured to simultaneously transmit at least some of the first signal to the third signal to the subject being examined.

According to another aspect of the present disclosure, a drowsiness warning method includes disposing a first sensor unit and a second sensor unit on eyelids of a subject being examined by using treatment equipment, measuring a distance between the first sensor unit and the second sensor unit, and comparing the measured distance with a predetermined distance, measuring a time for which the measured distance is maintained shorter than the predetermined distance, and comparing the measured time with the predetermined time, determining a drowsy state of the subject being examined based on the measured distance and the time, and when it is determined that the subject being examined is in a drowsy state, transmitting a drowsiness preventing signal to the subject being examined.

The drowsiness warning method may further include, when it is determined that the subject being examined is in the drowsy state, stopping, by a control unit, an operation of the treatment equipment.

The drowsiness warning method may further include, when it is determined that the subject being examined is in the drowsy state, generating, by a notification unit, a drowsiness notification signal for notifying an examiner.

The drowsiness warning signal may include at least one of a first signal including a sound signal, a second signal including an optical signal, or a third signal including a vibration signal.

The transmitting of the drowsiness preventing signal to the subject being examined may include transmitting the first to third signals to the subject being examined in a stepwise manner.

The transmitting of the drowsiness preventing signal to the subject being examined may include simultaneously transmitting at least some of the first to third signals to the subject being examined.

### Advantageous Effects of Disclosure

The drowsiness warning system and method according to the embodiments of the present disclosure may determine in real time whether the subject being examined under treatment is in the drowsy state by measuring the distance between the upper and lower eyelids of the subject being examined, and accordingly, prevent a posture change due to drowsiness of the subject being examined under treatment, thereby enhancing the accuracy of treatment and the effect of treatment.

### BRIEF DESCRIPTION OF DRAWINGS

FIG. 1 illustrates a drowsiness warning system according to an embodiment of the present disclosure.

FIG. 2 illustrate sensor units according to an embodiment of the present disclosure viewed when a subject being examined closes and opens his/her eyes.

FIG. 3 illustrate a method of transmitting a drowsiness preventing signal according to an embodiment of the present disclosure.

FIG. 4 illustrates a drowsiness warning system according to another embodiment of the present disclosure.

FIG. 5 illustrates a part of a drowsiness warning system according to another embodiment of the present disclosure.

FIG. 6 is a flowchart illustrating a drowsiness warning method according to an embodiment of the present disclosure.

### BEST MODE

The present disclosure may include various modifications and embodiments, and specific embodiments are illustrated in the drawings and are described in detail in the detailed description. However, it should be understood that the present disclosure is not limited to the specific embodiments, but includes all modifications, equivalents, and substitutes included in the spirit and technical scope of the present disclosure. In the following description of the present disclosure, a detailed description of known functions and configurations incorporated herein will be omitted when it may make the subject matter of the present disclosure rather unclear.

It will be understood that, although the terms first, second, etc. may be used herein to describe various components, these components should not be limited by these terms. The terms are used only for the purpose of distinguishing one component from other components.

The terminology used herein is for the purpose of describing particular embodiments only and is not intended to be limiting of the present disclosure. As used herein, the singular forms “a,” “an”, and “the” are intended to include the plural forms as well, unless the context clearly indicates otherwise. In addition, in each drawing, components are exaggerated, omitted, or schematically illustrated for convenience and clarity of description, and the size of each component is not entirely reflective.

In the description of each component, in the case where it is described as being formed “on” or “under”, “on” and “under” include all formed directly or with other components therebetween, and the criteria of “on” and “under” will be described with respect to the drawings.

Hereinafter, embodiments of the present disclosure will be described in detail with reference to the accompanying drawings, and in the description with reference to the accompanying drawings, the same or corresponding components are given the same reference numerals, and redundant descriptions thereof will be omitted.

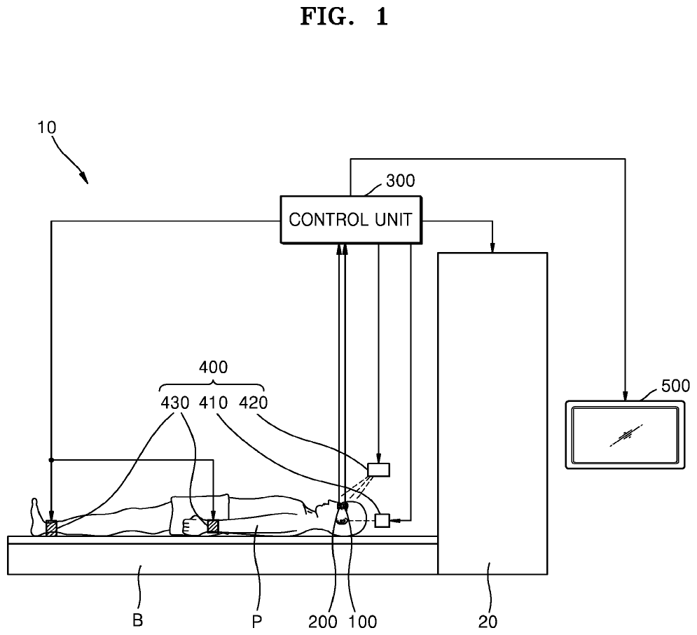

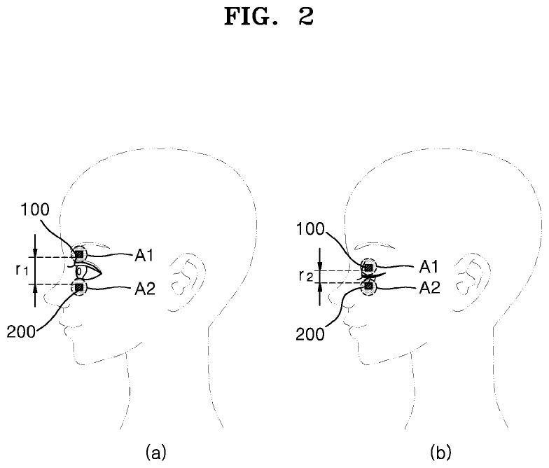

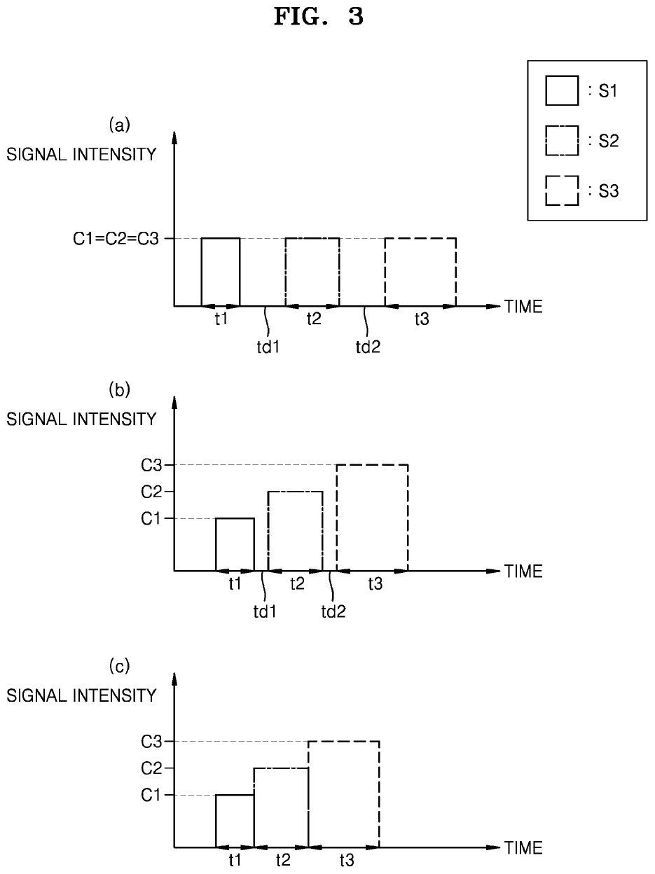

FIG. 1 illustrates a drowsiness warning system according to an embodiment of the present disclosure. FIG. 2 illustrate sensor units according to an embodiment of the present disclosure viewed when a subject being examined closes and opens his/her eyes. FIG. 3 illustrate a method of transmitting a drowsiness preventing signal according to an embodiment of the present disclosure.

Referring to FIGS. 1 to 3, a drowsiness warning system 10 may include a first sensor unit 100, a second sensor unit 200, a control unit 300, and an output unit 400. Also, the drowsiness warning system 10 may further include a notification unit 500.

The drowsiness warning system 10 may be a system including a device for preventing drowsiness of a subject being examined P while the subject being examined P receives treatment or examination by using treatment equipment 20. In this case, the treatment equipment 20 may be, for example, MRI or radiation treatment equipment. However, the present disclosure is not limited thereto, and the drowsiness warning system 10 according to the present disclosure may be applied to a field requiring prevention of drowsiness, such as a driver who is driving a vehicle, although not for therapeutic purposes.

The first sensor unit 100 may be mounted on any one part of the body of the subject being examined P, and in this case, the first sensor unit 100 may include a first sensor. The second sensor unit 200 may be mounted on another part of the body of the subject being examined P, and in this case, the second sensor unit 200 may include a second sensor. In this case, the first sensor unit 100 may measure a position of any one part of the body of the subject being examined P through the first sensor, and the second sensor unit 200 may measure a position of the other part of the body of the subject being examined P. The first sensor unit 100 and the second sensor unit 200 may be connected to the control unit 300, and may transmit the measured position of the body part of the subject being examined P to the control unit 300.

As an embodiment, the first sensor unit 100 may be mountable on any one of upper eyelids A1 and lower eyelids A2 of the subject being examined P, and the second sensor unit 200 may be mountable on the other one of the upper eyelids A1 and the lower eyelids A2 of the subject being examined P. However, for convenience of explanation, hereinafter, an embodiment in which the first sensor unit 100 may be mountable on the upper eyelids A1 of the subject being examined P, and the second sensor unit 200 may be mountable on the lower eyelids A2 of the subject being examined P will be mainly described.

The first sensor unit 100 may measure the position of the upper eyelids A1 of the subject being examined P, and the second sensor unit 200 may measure the position of the lower eyelids A2 of the subject being examined P. As an embodiment, the first sensor unit 100 may continuously and in real time measure the position of the upper eyelids A1 throughout a process of treating the subject being examined P by using the treatment equipment 20. At this time, the first sensor unit 100 may transmit the measured position of the upper eyelids A1 to the control unit 300. In addition, the second sensor unit 200 may continuously and in real time measure the position of the lower eyelids A2 throughout the process of treating the subject being examined P by using the treatment equipment 20. At this time, the second sensor unit 200 may transmit the measured position of the lower eyelids A2 to the control unit 300.

The control unit 300 may determine a drowsy state of the subject being examined P based on a distance between the first sensor unit 100 and the second sensor unit 200. At this time, the control unit 300 may be implemented, for example, in the form of a circuit board mounted on a computer for controlling the drowsiness warning system 10, a computer chip mounted on the circuit board, software embedded in the computer chip or embedded in the computer for controlling the drowsiness warning system 10, etc.

The control unit 300 may calculate the distance between the upper eyelids A1 and the lower eyelids A2 of the subject being examined P, based on the position of the first sensor unit 100 mounted on the upper eyelids A1 of the subject being examined P and the position of the second sensor unit 200 mounted on the lower eyelids A2 of the subject being examined P.

The control unit 300 may compare the calculated distance between the upper eyelids A1 and the lower eyelids A2 of the subject being examined P with a predetermined distance, and determine whether the distance between the upper eyelids A1 and the lower eyelids A2 is shorter than the predetermined distance. Hereinafter, for convenience of explanation, a state in which the distance between the upper eyelids A1 and the lower eyelids A2 of the subject being examined P is shorter than the predetermined distance is defined as an eyelids proximity state.

The control unit 300 may receive the positions of the upper eyelids A1 and the lower eyelids A2 respectively measured by the first sensor unit 100 and the second sensor unit 200, and continuously calculate the distance between the upper eyelids A1 and the lower eyelids A2 throughout the treatment process, thereby determining whether the subject being examined P is in the drowsy state. As an embodiment, the control unit 300 may measure a first distance r1 between the upper eyelids A1 and the lower eyelids A2 of the subject being examined P in an awake state when the subject being examined P starts treatment and a second distance r2 between the upper eyelids A1 and the lower eyelids A2 of the subject being examined P, which changes as treatment progresses. In this case, when the calculated second distance r2 is shorter than the predetermined distance, the control unit 300 may start measuring a time for which the distance between the upper eyelids A1 and the lower eyelids A2 is maintained as the second distance r2.

When the distance between the upper eyelids A1 and the lower eyelids A2 of the subject being examined P is shorter than the predetermined distance, the control unit 300 may measure a time for which the eyelids proximity state is maintained. In this regard, the control unit 300 may determine whether the time for which the upper eyelids A1 and the lower eyelids A2 of the subject being examined P are maintained in the eyelids proximity state is longer than the predetermined time. The control unit 300 may determine that the subject being examined P is in the drowsy state when the time for which the eyelids proximity state is maintained is longer than the predetermined time. As an embodiment, the predetermined time may be 2 seconds, and in this case, the control unit 300 may determine that the subject being examined P is in the drowsy state when the eyelids proximity state is maintained for 2 seconds or more.

The control unit 300 may be connected to the output unit 400. In this regard, when it is determined that the subject being examined P is in the drowsy state, the control unit 300 may control the output unit 400 to generate the drowsiness preventing signal.

The control unit 300 may be connected to the treatment equipment 20. In this case, the control unit 300 may stop an operation of the treatment equipment 20 when it is determined that the subject being examined P is in the drowsy state. As an embodiment, when the treatment equipment 20 is a radiation treatment equipment, when it is determined that the subject being examined P under treatment inside the radiation treatment equipment is in the drowsy state, the control unit 300 my stop the operation of the radiation treatment equipment to stop irradiating radiation to the subject being examined P and stop radiation therapy. As such, the radiation treatment is stopped when the subject being examined P under treatment is in the drowsy state, thereby preventing unnecessary radiation from being irradiated to a normal body part of the subject being examined P due to a change in a posture of the subject being examined P caused by the drowsy state. In addition, the control unit 300 may automatically stop the radiation treatment when the subject being examined P is in the drowsy state, and thus, there is no need to continuously monitor the state of the subject being examined P, thereby improving the convenience of examination and treatment.

The output unit 400 may generate the drowsiness preventing signal for preventing the subject being examined P from drowsing or for awakening the subject being examined P from the drowsy state. As an embodiment, the output unit 400 may generate the drowsiness preventing signal when the control unit 300 determines that the subject being examined P is in the drowsy state and induce the subject being examined P to be awakened from the drowsy state. As another embodiment, when an examiner monitoring the subject being examined P determines that the subject being examined P is to be in the drowsy state, the control unit 300 may control the output unit 400 to generate the drowsiness preventing signal so as to prevent the subject being examined P from being in the drowsy state. As described above, the effect of preventing the subject being examined P from falling into the drowsy state during treatment may be improved by the dual control including the control by the control unit 300 and the control by the examiner.

The output unit 400 may include a first signal generating unit 410 generating a first signal S1, a second signal generating unit 420 generating a second signal S2, and a third signal generating unit 430 generating a third signal S3. In this regard, the drowsiness preventing signal generated by the output unit 400 may include at least one of the first signal S1 to the third signal S3.

The first generating unit 410 may generate the first signal S1 for preventing the subject being examined P from drowsing. The first signal S1 may include, for example, a sound signal such as a warning sound, and as an embodiment, the first signal S1 may include a plurality of different types of sound signals. In this regard, the first generating unit 410 may be disposed on the head of the subject being examined P and its periphery (e.g., the periphery of the subject being examined P's ear). When the control unit 300 determines that the subject being examined P is in the drowsy state, the first signal S1 generated by the first generating unit 410 may be transmitted to the subject being examined P so that the subject being examined P may be awakened from the drowsy state. On the other hand, when the subject being examined P is not awakened from the drowsy state by the first signal S1, the control unit 300 may control the output unit 400 to change the type of the sound signal included in the first signal S1, the size of the signal, etc. and newly generate the first signal S1, and transmit the newly generated first signal S1 to the subject being examined P.

The second generating unit 420 may generate the second signal S2 for preventing the subject being examined P from drowsing. In this case, the second generating unit 420 may include illumination, and in this regard, the second signal S2 may include a light signal emitted by the illumination.

The second generating unit 420 may be disposed on the periphery of the head of the subject being examined P. When the control unit 300 determines that the subject being examined P is in the drowsy state, the second signal S2 generated by the second generating unit 420 may be transmitted to the subject being examined P so that the subject being examined P may be awakened. As an embodiment, the second generating unit 420 may awaken the drowsiness of the subject being examined P by emitting the generated light to the eyes of the subject being examined P in the drowsy state and peripheries thereof.

The second generating unit 420 may awaken the drowsiness of the subject being examined P by changing the intensity of light included in the second signal S2, the irradiation time, etc. and transmitting the same to the subject being examined P.

The third generating unit 430 may generate the third signal S3 for preventing the subject being examined P from drowsing. In this regard, the third signal S3 may be a signal for transmitting, to the subject being examined P, a shock for awakening the drowsy state of the subject being examined P. The third signal S3 may be, for example, a vibration signal, an electric shock signal, or a signal including all of the above signals. However, the present disclosure is not limited thereto, and the third signal S3 may include other types of signals capable of transferring a certain shock to the subject being examined P and awakening drowsiness. In this regard, the third generating unit 430 may change the intensity and period of the third signal S3 according to the drowsy state of the subject being examined P and transmit the third signal S3 to the subject being examined P.

The third generating unit 430 may be disposed on a part of the body of the subject being examined P. As an embodiment, the third generating unit 430 may be mounted on at least one of a wrist or an ankle of the subject being examined P. In this case, the third signal S3 generated by the third generating unit 430 may be transmitted to the wrist or the ankle of the subject being examined P so that the certain shock may be transferred to the subject being examined P, and thus, the subject being examined P may be awakened from drowsiness. As another embodiment, the third generating unit 430 may be mounted on an examination bed B supporting the subject being examined P. In this case, when the control unit 300 determines that the subject being examined P is in the drowsy state, the third signal S3 may be transmitted to the subject being examined P through the examination bed B (for example, the examination bed B is transferred in a shaking manner with a certain intensity and period), so that the drowsiness of the subject being examined P may be awakened.

The output unit 400 may transmit the generated drowsiness preventing signal to the subject being examined P. As an embodiment, the output unit 400 may transmit the first signal S1 to the third signal S3 to the subject being examined P in a stepwise manner as illustrated in FIG. 3. Here, transmitting in the stepwise manner may mean that sequentially transmitting the first signal S1, the second signal S2, and the third signal S3 to the subject being examined P, or transmitting the first signal S1, the second signal S2, and the third signal S3 to the subject being examined P while changing the transmission order and intensity of the first signal S1, the second signal S2, and the third signal S3.

For example, as shown in FIG. 3(a), the first signals S1 to S3 of the same intensity may be sequentially transmitted to the subject being examined P. In this regard, a first time t1 for which the first signal S1 is maintained, a second time t2 for which the second signal S2 is maintained, and a third time t3 for which the third signal S3 is maintained may be different from each other. For example, the first signal S1 to the third signal S3 of the same intensity may be sequentially transmitted to the subject being examined P, and, simultaneously, the first time t1 to the third time t3 may gradually increase. However, the present disclosure is not limited thereto. The order in which the first signal S1 to the third signal S3 are transmitted may be changed, or the first time t1 to the third time t3 may be the same.

As another example, as shown in FIG. 3(b), an intensity C1 of the first signal S1 injected into the subject being examined P, an intensity C2 of the second signal S2, and an intensity C3 of the third signal S3 may be different from each other. For example, the first signal S1 to the third signal S3 may be sequentially transmitted to the subject being examined P while the intensity C1 of the first signal S1, the intensity C2 of the second signal S2, and the intensity C3 of the third signal S3 gradually increase. In this case, a first interval td1 may exist between a time at which the first signal S1 ends and a time at which the second signal S2 starts, and a second interval td2 may exist between a time at which the second signal S2 ends and a time at which the third signal S3 starts. In this case, the first interval td1 and the second interval td2 may be the same as each other. However, the present disclosure is not limited thereto, and the first interval td1 and the second interval td2 may be different from each other. In addition, the order in which the first signal S1 to the third signal S3 are transmitted may be changed, or the first time t1 to the third time t3 may be the same as each other.

As another example, as shown in FIG. 3(c), there is no interval between the time at which the first signal S1 ends and the time at which the second signal S2 starts, and between the time at which the second signal S2 ends and the time at which the third signal S3 starts, and the first signal S1 to the third signal S3 may be continuously transmitted to the subject being examined P. As described above, the order in which the first signal S1 to the third signal S3 are transmitted may be changed, and the first time t1 to the third time t3 may be the same as each other.

When it is determined that the subject being examined P is in the drowsy state even after transmitting the first signal S1 to the third signal S3 to the subject being examined P, the control unit 300 may control the output unit 400 to generate a new additional signal, and the output unit 400 may transmit the additional signal to the subject being examined P.

As described above, the output unit 400 may transmit the first signal S1 to the third signal S3 in the stepwise manner to the subject being examined P, and apply stimulus (or shocks) of different types and/or strengths by signals of different types and/or intensities in the stepwise manner to the subject being examined P in the drowsy state, and thus, the subject being examined P may be certainly released from the drowsy state. In addition, even when the subject being examined P in the drowsy state adapts to the stimulus by any one signal and does not awaken from the drowsy state, the output unit 400 may transmit signals of different types and/or intensities to the subject being examined P, thereby enhancing the effect of awakening the drowsiness of the subject being examined P.

As another embodiment, the output unit 400 may transmit at least some of the first signal S1, the second signal S2, and the third signal S3 to the subject being examined P simultaneously. For example, the output unit 400 may transmit the first signal S1 to the third signal S3 to the subject being examined P simultaneously and awaken the subject being examined P from drowsiness.

As another example, the output unit 400 may simultaneously transmit two of the first signal S1 to the third signal S3 to the subject being examined P. For example, the output unit 400 may transmit the first signal S1 and the second signal S2 to the subject being examined P simultaneously for a certain time, and then, when it is determined that the subject being examined P is still in the drowsy state, simultaneously transmit the second signal S2 and the third signal S3 to the subject being examined P, thereby awakening the drowsiness of the subject being examined P. In this case, types of the two signals transmitted together may be changed. As another embodiment, the output unit 400 may generate an additional signal other than the first signal S1 to the third signal S3. In this case, the output unit 400 may simultaneously transmit three or more signals to the subject being examined P and awaken the subject being examined P from drowsiness.

As described above, the output unit 400 may generate and combine a plurality of different signals and transmit the signals to the subject being examined P, thereby enhancing the effect of awakening the drowsiness of the subject being examined P compared to using only a single signal.

The notification unit 500 may notify an examiner (not shown) of the drowsy state of the subject being examined P. Here, the examiner may mean a person who controls the treatment equipment 20 to treat the subject being examined P or monitors the subject being examined P to be treated by the treatment equipment 20.

The notification unit 500 may be connected to the control unit 300 to receive information about whether the subject being examined P is in the drowsy state from the control unit 300. In this regard, when it is determined that the subject being examined P is in the drowsy state, the control unit 300 may control the notification unit 500 to generate a drowsiness notification signal for notifying the examiner of the drowsy state of the subject being examined P. The notification unit 500 may be, for example, a sound device capable of generating a notification sound or a display device that visually displays a state of a patient. Also, the notification unit 500 may be a device including both a sound unit and a display unit.

By the drowsiness notification signal generated by the notification unit 500, the examiner may confirm that the subject being examined P under treatment is in the drowsy state. In this case, the examiner may awaken the drowsiness of the subject being examined P by stopping the operation of the treatment equipment 20 or directly controlling the output unit 400 to transmit the drowsiness preventing signal to the subject being examined P. Thereby, through the dual control method including the control of the output unit 400 and/or the treatment equipment 20 by the control unit 300 and the control of the output unit 400 and/or the treatment equipment 20 by the examiner, the effect of preventing the subject being examined P from falling into the drowsy state during treatment may be improved, and unnecessary radiation may be prevented from being irradiated to the normal body part of the subject being examined P.

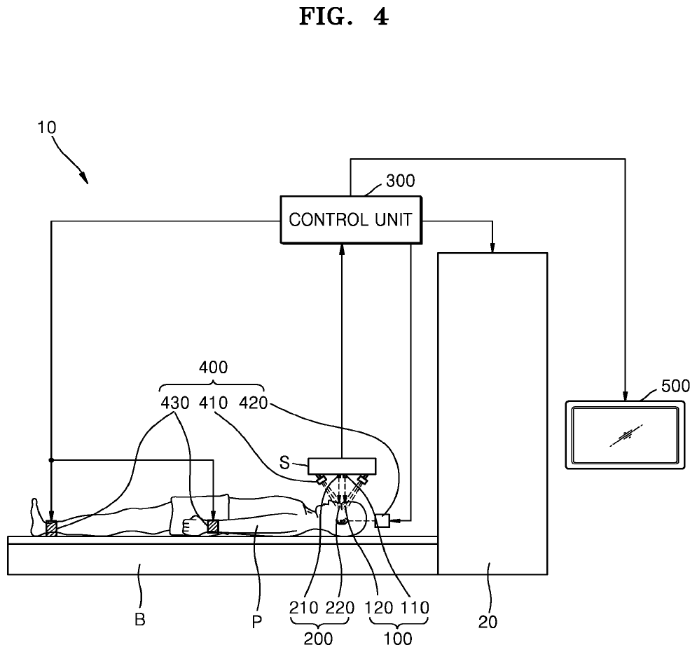

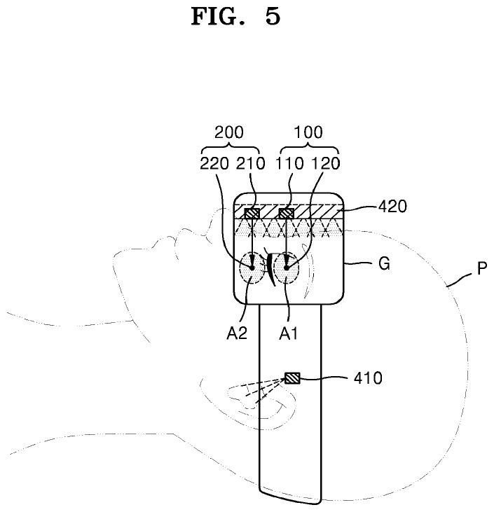

FIG. 4 illustrates a drowsiness warning system according to another embodiment of the present disclosure. FIG. 5 illustrates a part of a drowsiness warning system according to another embodiment of the present disclosure.

Referring to FIGS. 4 and 5, the drowsiness warning system 10 may include the first sensor unit 100, the second sensor unit 200, the control unit 300, and the output unit 400. Also, the drowsiness warning system 10 may further include the notification unit 500.

The first sensor unit 100 may include a first sensor 110 and a first marker 120. The first sensor 110 may detect a position of the first marker 120. The second sensor unit 200 may include a second sensor 210 and a second marker 220. The second sensor 210 may detect a position of the second marker 220.

The first sensor 110 may be disposed to be spaced apart from the subject being examined P, and the first marker 120 may be mountable on any one of the upper eyelids A1 and the lower eyelids A2 of the subject being examined P. In this regard, the second sensor 210 may be disposed to be spaced apart from the subject being examined P, and the second marker 220 may be mountable on the other one of the upper eyelids A1 and the lower eyelids A2 of the subject being examined P. However, hereinafter, for convenience of explanation, an embodiment in which the first marker 120 may be mountable on the upper eyelids A1 of the subject being examined P, and the second marker 220 may be mountable on the lower eyelids A2 of the subject being examined P will be mainly described.

The first sensor 110 may measure the position of the first marker 120 mounted on the upper eyelids A1 of the subject being examined P, and the second sensor 210 may measure the position of the second marker 220 mounted on the lower eyelids A2 of the subject being examined P. The first sensor 110 and the second sensor 210 may transmit the measured positions of the first marker 120 and the second marker 220 to the control unit 300. In this case, based on the received positions of the first marker 120 and the second marker 220, the control unit 300 may calculate a distance between the upper eyelids A1 and the lower eyelids A2 of the subject being examined P. In this regard, determining that the subject being examined P is in a drowsy state when the calculated distance between the upper eyelids A1 and the lower eyelids A2 of the subject being examined P is shorter than a predetermined distance is the same as described above, and thus, a redundant description thereof is omitted.

In an embodiment, the first sensor 110 and the second sensor 210 may be disposed on a support S. In this case, the support S is spaced apart from the subject being examined P, so that the first sensor 110 and the second sensor 210 may be respectively spaced apart from the first marker 120 and the second marker 220 mounted on the subject being examined P. Meanwhile, the second generating unit 420 may be disposed on the support S. In this case, the second generating unit 420 may generate the second signal S2 and irradiate light onto the periphery of the eyelids of the subject being examined P to awaken drowsiness of the subject being examined P.

The first sensor 110 and the second sensor 210 may be respectively spaced apart from the first marker 120 and the second marker 220 mounted on the subject being examined P. Meanwhile, the second generating unit 420 may be disposed on the support S. In this case, the second generating unit 420 may generate the second signal S2 and irradiate light included in the second signal S2 onto the periphery of the eyelids of the subject being examined P to awaken the drowsiness of the subject being examined P.

As another embodiment, the first sensor 110 and the second sensor 210 may be disposed on a detachable support G. In this case, the detachable support G may be detachably disposed on the head of the subject being examined P. The detachable support G may be, for example, in the form of goggles.

The first sensor 110 and the second sensor 210 may be disposed on the inner surface of the detachable support G. In this regard, the first sensor 110 may be disposed on a part of the inner surface of the detachable support G facing the upper eyelids A1 when the subject being examined P wears the detachable support G, and the second sensor 210 may be disposed on another part of the inner surface of the detachable support G facing the lower eyelids A2 when the subject being examined P wears the detachable support G. In a state in which the subject being examined P wears the detachable support G, the first sensor 110 may measure the position of the first marker 120, and the second sensor 210 may measure the positon of the second marker 220.

The detachable support G may include a fixing unit. The fixing unit may fix the detachable support G and the head of the subject being examined P. In this regard, the first signal generating unit 410 may be disposed on the fixing unit of the detachable support G. In an embodiment, the first generating unit 410 may be disposed on the fixing unit so as to be adjacent to the ear of the subject being examined P and its periphery. In this case, when it is determined by the control unit 300 that the subject being examined P is in the drowsy state, the first signal generating unit 410 may generate and transmit the first signal S1 including a warning sound, etc. to the ear of the subject being examined P wearing the detachable support G and its periphery.

The second signal generating unit 420 may be disposed on the inner surface of the detachable support G. In this case, when it is determined by the control unit 300 that the subject being examined P is in the drowsy state, the second signal generating unit 420 may generate and transmit the second signal S2 to the eye of the subject being examined P wearing the detachable support G and its periphery.

In addition, the third signal generating unit 430 may be disposed on the detachable support G. In this case, when it is determined by the control unit 300 that the subject being examined P is in the drowsy state, the third signal generating unit 430 may generate and transmit the third signal S3 such as vibration, etc. to the head of the subject being examined P wearing the detachable support G, thereby awakening the drowsiness of the subject being examined P.

The sensor units 100 and 200 and the output unit 400 are stably fixed to the subject being examined P by the detachable support G as described above, thereby preventing a decrease in the accuracy of measuring whether the subject being examined P is in the drowsy state by using the first sensor unit 100 and the second sensor unit 200 due to the change in the posture of the subject being examined P caused by the movement of the subject being examined P during treatment. In addition, even when a body part requiring treatment is not a front body part of the subject being examined P but is located on the side or rear of the subject being examined P, the influence according to the posture of the subject being examined P may be minimized, thereby accurately determining whether the subject being examined P is in the drowsy state.

Specific characteristics of the control unit 300, the output unit 400, and the notification unit 500 are the same as those described above, and thus redundant descriptions thereof are omitted.

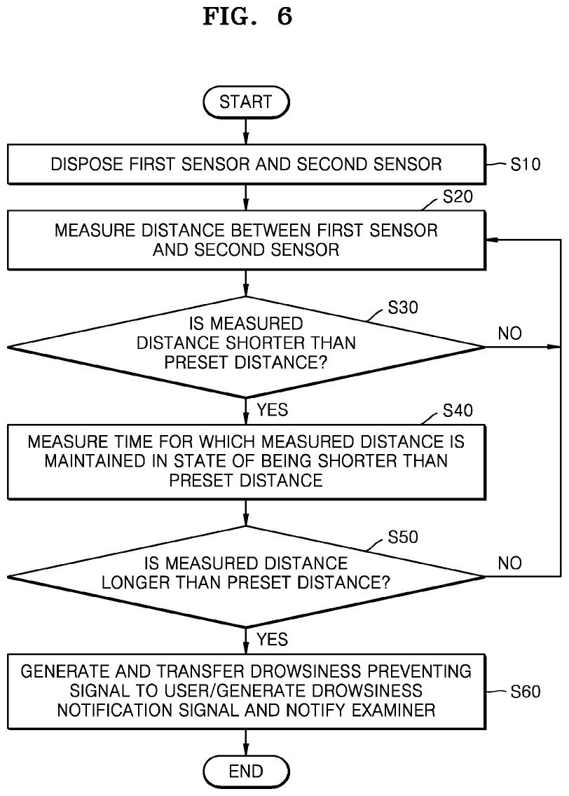

FIG. 6 is a flowchart illustrating a drowsiness warning method according to an embodiment of the present disclosure.

Referring to FIG. 6, the drowsiness warning method using the drowsiness warning system 10 may be as follows, and for convenience of explanation, an embodiment in which the first sensor unit 100 and the second sensor unit 200 may be respectively mountable on the upper eyelids A1 and the lower eyelids A2 will be mainly described.

First, the first sensor unit 100 may be mounted on the upper eyelids A1 of the subject being examined P, and the second sensor unit 200 may be mounted on the lower eyelids A2 of the subject being examined P (S10). In this case, the first sensor unit 100 may measure the position of the upper eyelids A1 and transmit the position to the control unit 300, and the second sensor unit 200 may measure the position of the lower eyelids A2 and transmit the position to the control unit 300.

Next, the control unit 300 may calculate the distance between the upper eyelids A1 and the lower eyelids A2 of the subject being examined P based on the received positions of the upper eyelids A1 and the lower eyelids A2.

Next, the control unit 300 may determine whether the calculated distance between the upper eyelids A1 and the lower eyelids A2 is shorter than a predetermined distance (S30). As an embodiment, when it is determined that the distance between the upper eyelids A1 and the lower eyelids A2 of the subject being examined P is shorter than the predetermined distance (i.e., the eyelids proximity state), the control unit 300 may measure a time for which the eyelids proximity state is maintained (S40). As another embodiment, when it is determined that the distance between the upper eyelids A1 and the lower eyelids A2 of the subject being examined P is longer than the predetermined distance, the control unit 300 may determine that the subject being examined P is awakened so that treatment may be continued.

Next, when the distance between the upper eyelids A1 and the lower eyelids A2 of the subject being examined P is shorter than the predetermined distance, the control unit 300 may determine whether the measured eyelids proximity state maintenance time is longer than the predetermined time (S50).

As an embodiment, when the measured eyelids proximity state maintenance time is longer than the predetermined time, the control unit 300 may determine that the subject being examined P is in a drowsy state. At this time, the control unit 300 may control the output unit 400 to generate a drowsiness preventing signal, and accordingly, the output unit 400 may generate the drowsiness preventing signal and transmit the drowsiness preventing signal to the subject being examined P in the drowsy state, thereby awakening drowsiness of the subject being examined P. On the other hand, the control unit 300 may control the notification unit 500 to generate a drowsiness notification signal for notifying an examiner that the subject being examined P is in the drowsy state, and accordingly, the notification unit 500 may generate the drowsiness notification signal and transmit the drowsiness notification signal to the examiner. As another embodiment, when the measured eyelids proximity state maintenance time is shorter than the predetermined time, the control unit 300 may determine that the subject being examined P is in an awake state so that treatment may be continued.

As described above, the drowsiness warning system 10 and method according to embodiments of the present disclosure may determine in real time whether the subject being examined P is in the drowsy state by measuring the distance between the upper eyelids A1 and the lower eyelids A2 of the subject being examined P, thereby preventing the change in posture due to the drowsiness of the subject being examined P under treatment, and improving the accuracy of treatment and the effect of treatment.

While the present disclosure has been particularly shown and described with reference to embodiments thereof, it will be understood by one of ordinary skill in the art that various changes and equivalent other embodiments may be made therein. Accordingly, the spirit and scope of the present disclosure are defined by the following claims.

1. A drowsiness warning system comprising:
a first sensor unit configured to be mountable on any one of upper eyelids or lower eyelids of a subject being examined under treatment by using treatment equipment;
a second sensor unit configured to be mountable on the other one of the upper eyelids or the lower eyelids of the subject being examined;
an output unit configured to generate a drowsiness preventing signal for preventing drowsiness of the subject being examined; and
a control unit configured to determine a drowsy state of the subject being examined, based on a distance between the first sensor unit and the second sensor unit,
wherein, when the distance is maintained for a predetermined time or more in a state of being shorter than a predetermined distance, the control unit is further configured to determine that the subject being examined is in the drowsy state, and control the output unit to transmit a drowsiness preventing signal to the subject being examined.

2. The drowsiness warning system of claim 1, wherein
the control unit is connected to the treatment equipment and is further configured to stop an operation of the treatment equipment when it is determined that the subject being examined is in the drowsy state.

3. The drowsiness warning system of claim 1, further comprising:
a notification unit configured to notify an examiner of the drowsy state of the subject being examined,
Wherein, when it is determined that the subject being examined is in the drowsy state, the control unit is further configured to control the notification unit to generate a drowsiness warning signal for notifying the examiner.

4. The drowsiness warning system of claim 1, wherein
the drowsiness warning signal comprises at least one of
a first signal comprising a sound signal;
a second signal comprising an optical signal; or
a third signal comprising a vibration signal.

5. The drowsiness warning system of claim 4, wherein
the output unit is further configured to transmit the first to third signals to the subject being examined in a stepwise manner.

6. The drowsiness warning system of claim 5, wherein
the output unit is further configured to simultaneously transmit at least some of the first signal to the third signal to the subject being examined.

7. A drowsiness warning method comprising:
disposing a first sensor unit and a second sensor unit on eyelids of a subject being examined by using treatment equipment;
measuring a distance between the first sensor unit and the second sensor unit, and comparing the measured distance with a predetermined distance;
measuring a time for which the measured distance is maintained shorter than the predetermined distance, and comparing the measured time with the predetermined time;
determining a drowsy state of the subject being examined based on the measured distance and the time; and
when it is determined that the subject being examined is in a drowsy state, transmitting a drowsiness preventing signal to the subject being examined.

8. The drowsiness warning method of claim 7, further comprising:
when it is determined that the subject being examined is in the drowsy state, stopping, by a control unit, an operation of the treatment equipment.

9. The drowsiness warning method of claim 7, further comprising:
when it is determined that the subject being examined is in the drowsy state, generating, by a notification unit, a drowsiness notification signal for notifying an examiner.

10. The drowsiness warning method of claim 7, wherein
the drowsiness warning signal comprises at least one of
a first signal comprising a sound signal;
a second signal comprising an optical signal; or
a third signal comprising a vibration signal.

11. The drowsiness warning method of claim 10, wherein
the transmitting of the drowsiness preventing signal to the subject being examined comprises transmitting the first to third signals to the subject being examined in a stepwise manner.

12. The drowsiness warning method of claim 10, wherein
the transmitting of the drowsiness preventing signal to the subject being examined comprises simultaneously transmitting at least some of the first to third signals to the subject being examined.
